(19) United States
(12) Plant Patent Application Publication
(10) Pub. No.: US 2019/0380242 P1
(43) Pub. Date: Dec. 12, 2019

(54) Strawberry plant variety named 'Drisstrawfiftynine'

(50) ;

(71) Applicant: Driscoll's, Inc., Watsonville, CA (US)

(72) Inventors: Katalin Monika Pakozdi, Maidstone (GB); Carlos D. Fear, Aptos, CA (US); Alessandra Lillo, Watsonville, CA (US)

(73) Assignee: Driscoll's, Inc., Watsonville, CA (US)

(21) Appl. No.: 15/998,015

(22) Filed: Jun. 11, 2018

(51) Int. Cl. A01H 6/74

(52) U.S. Cl. PLT/208

(57) ABSTRACT

A new and distinct variety of strawberry plant named ‘DrisStrawFiftyNine’, particularly characterized by its yield potential, fruit with less seed, fruit size, fruit appearance, and fruit shelf-life, is disclosed.

Latin name: Botanical classification: Fragaria x ananassa.

Varietal denomination: The varietal denomination of the claimed variety of strawberry plant is ‘DrisStrawFiftyNine’.

### BACKGROUND OF THE INVENTION

Cultivated strawberry is a hybrid species of the genus Fragaria that is grown worldwide for its fruit. Modern strawberry was first bred in Brittany, France, in the 18^{th} century by crossing Fragaria virginiana with Fragaria chiloensis. Strawberry fruit is an aggregate accessory fruit, with the fleshy part of the fruit being derived from the receptacle that holds the ovaries.

Strawberry varieties vary widely in color, size, shape, flavor, season of ripening, degree of fertility, and susceptibility to disease. Certain varieties vary in foliage, and some vary in the relative development of their reproductive organs. Typically, strawberry flowers appear hermaphroditic in structure, but function as either male or female. Generally, commercial production of strawberry plants involves propagation from runners and distribution as either plugs or bare root plants. Cultivation is either perennial or annual plasticulture. During the off season, strawberries can also be produced in greenhouses.

Strawberry fruit is widely appreciated for its characteristic bright red color, aroma, juicy texture, and sweetness. Strawberry fruit is a popular fruit that is generally consumed either fresh or in prepared foods, such as preserves and baked goods.

Strawberry is an important and valuable fruit crop. Accordingly, there is a need for new varieties of strawberry plants. In particular, there is a need for improved varieties of strawberry plant that are stable, high yielding, and agronomically sound.

### SUMMARY OF THE INVENTION

In order to meet these needs, the present invention is directed to an improved variety of strawberry plant. In particular, the invention relates to a new and distinct variety of strawberry plant (Fragaria x ananassa), which has been denominated as ‘DrisStrawFiftyNine’.

Strawberry plant variety ‘DrisStrawFiftyNine’ was discovered in East Malling, Kent County, the United Kingdom in August of 2012, and originated from a cross between the proprietary female parent ‘UUKE 116-003’ (unpatented) and the proprietary male parent ‘UUKE 167-027’ (unpatented). A single plant was selected and asexually propagated via stolons in the Netherlands in August of 2012.

‘DrisStrawFiftyNine’ was subsequently asexually propagated via stolons, and underwent further testing at a farm in East Malling, Kent County, the United Kingdom for five years (2012 to 2016). The present variety has been found to be stable and reproduce true to type through successive asexual propagations via stolons.

‘DrisStrawFiftyNine’ exhibits the following distinguishing characteristics when grown under normal horticultural practices in East Malling, Kent County, the United Kingdom:

1. High yield; and

2. Fruit having less seed, better appearance, and improved shelf-life.

‘DrisStrawFiftyNine’ was selected for its yield potential, fruit with less seed, fruit size, fruit appearance, and fruit shelf-life.

### DESCRIPTION OF THE DRAWINGS

This new strawberry plant is illustrated by the accompanying photographs which show fruit and leaves of the plant. The colors shown are as true as can be reasonably obtained by conventional photographic procedures. The photographs are of plants that are six months old.

FIG. 1 illustrates whole fruit of variety ‘DrisStrawFiftyNine’.

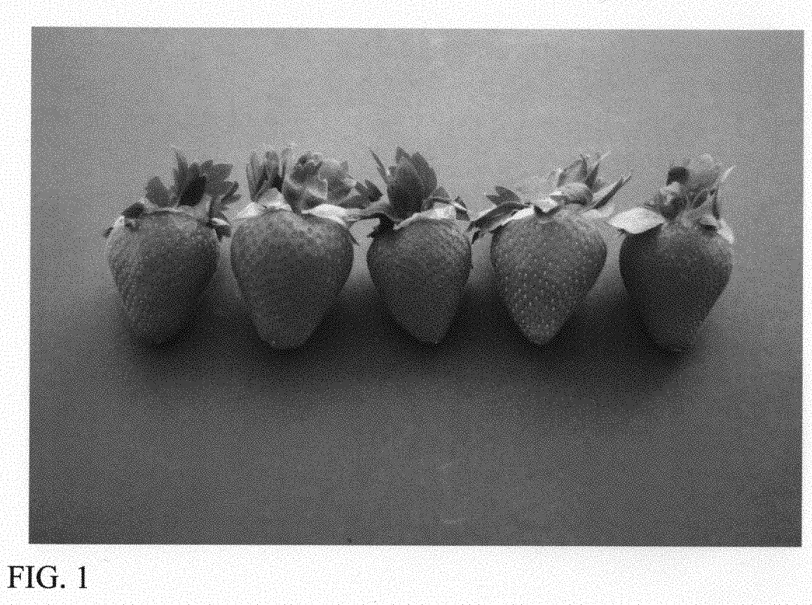

FIG. 2A illustrates the upper surface of a leaf of variety ‘DrisStrawFiftyNine’. FIG. 2B illustrates the lower surface of a leaf of variety ‘DrisStrawFiftyNine’.

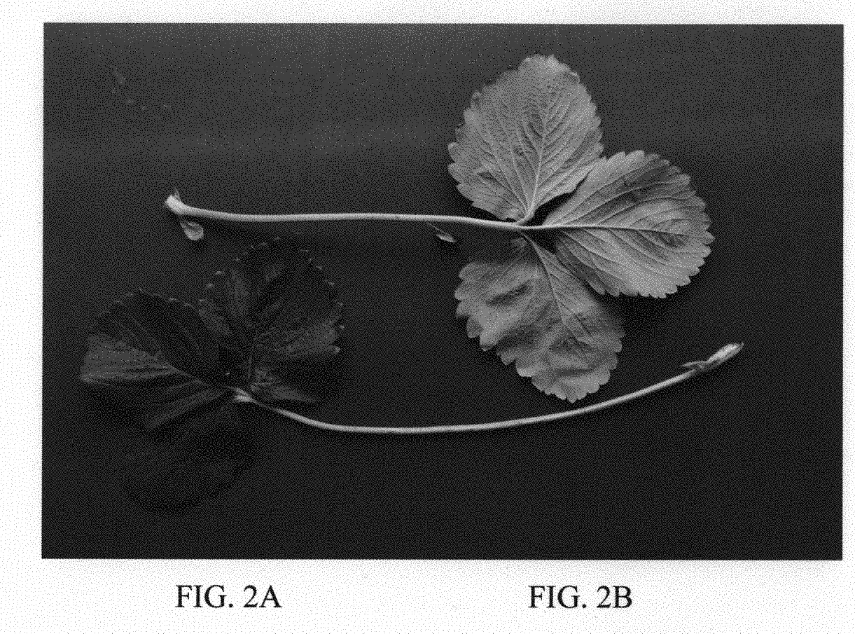

### DESCRIPTION OF THE NEW VARIETY

The following detailed descriptions set forth the distinctive characteristics of ‘DrisStrawFiftyNine’. The data which define these characteristics is based on observations taken in East Malling, Kent County, the United Kingdom from 2012 to 2016. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawFiftyNine’ has not been observed under all possible environmental conditions. The botanical description of ‘DrisStrawFiftyNine’ was taken from plants that were six months old. The indicated values represent averages calculated from measurements of several plants. Color references are primarily to The R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
   Species.—Fragaria x ananassa.
   Common name.—Strawberry.
   Denomination.—‘DrisStrawFiftyNine’.
Parentage:
   Female parent.—The proprietary variety ‘UUKE 116-003’ (unpatented).
   Male parent.—The proprietary variety ‘UUKE 167-027’ (unpatented).
Plant:
   Height.—39.7 cm.
   Diameter.—45.0 cm.
   Number of crowns/plant.—5.
   Growth habit.—Semi-upright.
Stolon:
   Average number of daughter plants per square foot.—44.
   Diameter at bract.—4.794 mm.
   Anthocyanin coloration.—Absent to medium.
   Average length.—47.8 cm.
   Surface texture.—Glabrous.
   Color.—RHS 145A (Strong yellow-green).
Leaf:
   Number of leaflets.—Three only.
   Color of upper surface.—RHS NN137B (Greyish olive green).
   Color of lower surface.—RHS NN137D (Greyish olive green).
   Leaf length.—37.5 cm.
   Leaf width.—19 cm.
   Blistering.—Absent or weak.
   Glossiness.—Medium.
   Variegation.—Absent.
   Terminal Leaflets.—Length: 10.9 cm. Width: 9.1 cm. Length/width ratio: 1.2. Number of teeth/terminal leaflet: 26. Shape of base: Obtuse. Margin: Crenate. Shape in cross section: Concave.
   Petiole.—Length: 26.0 cm. Diameter: 6.08 mm. Attitude of hairs: Horizontal. Bract frequency (number present on each petiole): 1. Petiole color: RHS 145A (Strong yellow-green).
   Petiolule.—Length: 11.12 mm. Diameter: 2.78 mm. Petiolule color: RHS 145B (Light yellow-green).
   Stipule.—Length: 3.9 cm. Width: 8.68 mm. Stipule color: RHS N144D (Strong yellow-green). Anthocyanin coloration: Absent.
Inflorescence:
   Position in relation to foliage.—Above.
   Pedicel.—Attitude of hairs: Upwards.
   Flower.—Flower diameter (petal tip to petal tip on non-flattened flower): 34.46 mm. Arrangement of petals: Overlapping. Stamen: Present. Typical and observed number of flowers per plant: 28.
   Petal.—Length: 13.37 mm. Width: 16.14 mm. Length/width ratio: 0.8. Typical and observed petal number: 6. Color of upper side: RHS NN155C (White). Color of lower side: RHS NN155C (White). Apex shape: Rounded. Base shape: Obtuse.
   Calyx.—Diameter (sepal tip to sepal tip, measured on back of flower): 44.49 mm. Size of calyx in relation to corolla: Larger.
   Sepal.—Length (sepal tip to point of attachment to receptacle): 17.42 mm. Width: 10.40 mm. Typical and observed sepal number: 6. Sepal color: RHS N134A (Dark yellowish green). Apex shape: Truncate/emarginate. Base shape: Orbicular.
Fruit:
   Length.—48.69 mm.
   Width.—40.62 mm.
   Length/width ratio.—1.2.
   Fruit hollow length.—26.30 mm.
   Fruit hollow width.—16.38 mm.
   Fruit hollow length/width ratio.—1.6.
   Shape.—Conical.
   Color.—RHS 44A (Vivid red).
   Position of achenes.—Level with surface.
   Position of calyx attachment.—Level with fruit.
   Diameter of calyx in relation to diameter of fruit.—Much larger.
   Attitude of sepals.—Upwards.
   Color of flesh (excluding core).—RHS 40B (Vivid reddish orange).
   Color of core.—RHS 41C (Moderate reddish orange).
   Evenness of color.—Even or very slightly uneven.
   Glossiness.—Medium.
   Evenness of surface.—Even or very slightly uneven.
   Width of band without achenes.—Absent or very narrow.
   Firmness.—Medium.
   Cavity.—Absent or small.
   Brix percentage.—9.1%.
   Average number of achenes per fruit.—322.
   Fruit market use.—Fresh consumption.
   Fruit shelf life.—longer shelf life as compared to fruit of female parent ‘UUKE 116-003’.
   Fruiting truss.—Length: 35.7 cm. Diameter: 5.66 mm. Number of berry per fruiting truss: 9.
Production:
   Flowering interval.—May to September.
   Harvest interval.—Mid-May to early September.
   Type of bearing.—Fully remontant.
   Productivity.—1.023 kg to 1.050 kg of fruit per season from two-month-old to six-month-old plants when grown in East Malling, Kent County, the United Kingdom.
Resistance to diseases:
   Powdery mildew (Podosphaera macularis).—Moderately resistant.
   Verticillium wilt.—Moderately susceptible.

### COMPARISON WITH PARENTAL AND COMMERCIAL VARIETIES

‘DrisStrawFiftyNine’ differs from the proprietary female parent ‘UUKE 116-003’ (unpatented) in that plants of ‘DrisStrawFiftyNine’ produce higher yields of fruit as compared to plants of ‘UUKE 116-003’. Additionally, fruit of ‘DrisStrawFiftyNine’ are less seedy, larger in size, better in appearance, and have a longer shelf-life as compared to fruit of ‘UUKE 116-003’.

‘DrisStrawFiftyNine’ differs from the proprietary male parent ‘UUKE 167-027’ (unpatented) in that plants of ‘DrisStrawFiftyNine’ produce higher yields of fruit as compared to plants of ‘UUKE 167-027’. Additionally, fruit of ‘DrisStrawFiftyNine’ are smaller in size as compared to fruit of “UUKE 167-027’.

‘DrisStrawFiftyNine’ differs from the commercial variety ‘DrisStrawThirtyFive’ (U.S. Plant Pat. No. 24,745) in that plants of ‘DrisStrawFiftyNine’ have an upright growth habit, while plants of ‘DrisStrawThirtyFive’ have a semi-upright growth habit. Additionally, plants of ‘DrisStrawFiftyNine’ have medium vigor, while plants of ‘DrisStrawThirtyFive’ have strong vigor. Moreover, the type of bearing of ‘DrisStrawFiftyNine’ is fully remontant, while the type of bearing of ‘DrisStrawThirtyFive’ is not remontant.

‘DrisStrawFiftyNine’ differs from the commercial variety ‘DrisStrawTwo’ (U.S. Plant Pat. No. 18,878) in that terminal leaflets of ‘DrisStrawFiftyNine’ have an obtuse shape of base, while terminal leaflets of ‘DrisStrawTwo’ have a rounded shape of base. Additionally, fruit of ‘DrisStrawFiftyNine’ have absent or a very narrow width of band without achenes, while fruit of ‘DrisStrawTwo’ have a medium width of band without achenes. Moreover, fruit of ‘DrisStrawFiftyNine’ have a calyx attachment that is level with fruit, while fruit of ‘DrisStrawTwo’ have a calyx attachment that is raised from fruit.

We claim:

1. A new and distinct variety of strawberry plant named ‘DrisStrawFiftyNine’ as shown and described herein.
